(19) United States
(12) Patent Application Publication
(10) Pub. No.: US 2022/0406429 A1
(43) Pub. Date: Dec. 22, 2022

(54) Artificial Intelligence Enabled Control of Hemodynamics in Surgery Patients

(71) Applicant: The Regents of the University of California, Oakland, CA (US)

(72) Inventors: Chih-Ming Ho, Los Angeles, CA (US); Daniel Garcia, Los Angeles, CA (US); Jinyoung Brian Jeong, Los Angeles, CA (US); Jacques Neelankavil, Los Angeles, CA (US); Jure Marijic, Los Angeles, CA (US); Soban Umar, Oakland, CA (US); Michael Zargari, Oakland, CA (US)

(73) Assignee: The Regents of the University of California, Oakland, CA (US)

(21) Appl. No.: 17/755,669

(22) PCT Filed: Nov. 4, 2020
(86) PCT No.: PCT/US2020/058940

§ 371 Date: May 4, 2022

Related U.S. Application Data
(60) Provisional application No. 62/930,378, filed on Nov. 4, 2019.

(51) Int. Cl. G16H 20/10; A61P 9/08; A61P 9/12; A61B 5/021

(52) U.S. Cl. CPC G16H 20/10; A61P 9/08; A61P 9/12; A61B 5/021

(57) ABSTRACT

Systems and methods for artificial intelligence enable control of hemodyamics in an individual are provided. A number of embodiments use a phenotypic response surface (PRS) that describes a physiological response to a vasopressor and/or a vasodilator to guide administration of the vasopressor and/or vasodilator. Various embodiments continually update the PRS based on changes in physiological response to the vasopressor and/or vasodilator.

### CROSS REFERENCE TO RELATED APPLICATIONS

This application claims priority to U.S. Provisional Application Ser. No. 62/930,378, entitled “Artificial Intelligence Enabled Control of Hemodynamics and Anesthesia in Surgery Patients,” filed Nov. 4, 2019, which is hereby incorporated by reference in its entirety.

### FIELD OF THE INVENTION

The present invention generally relates to systems and methods for artificial intelligence enabled control of hemodyamics and anesthesia in surgery patients.

### BACKGROUND OF THE INVENTION

Precise management of the hemodynamics of patients undergoing surgery and in the Intensive Care Unit (ICU) is challenging due to a myriad of patient-specific factors. Medications are typically selected to manage hemodynamics based on the medication pharmacokinetics and the dosing is intermittently modified to obtain a desired therapeutic effect. The response of patients to different medications, such as vasopressors that are used to elevate blood pressure, is complicated by patients' unique and dynamic physiologic responses to medications that can evolve throughout the course of their surgery or hospitalization.

### BRIEF SUMMARY OF THE INVENTION

Many embodiments are directed to systems and methods for artificial intelligence enabled control of hemodyamics and anesthesia in surgery patients and applications thereof.

In one embodiment, a method for artificial intelligence enabled control of hemodyamics includes administering at least one of a vasopressor or a blood pressure reducing agent to an individual, constructing a phenotypic response surface (PRS) describing the physiologic response of the individual in reaction to the at least one of the vasopressor or the blood pressure reducing agent, and controlling the blood pressure of the individual by administering at least one dose of the vasopressor or the blood pressure reducing agent based on the physiological response described in the PRS.

In a further embodiment, the method further includes updating the PRS based on additional physiological response to the at least one of the vasopressor or the blood pressure reducing agent.

In another embodiment, the administering step is provides a set number of doses of the at least one of the vasopressor or the blood pressure reducing agent.

In a still further embodiment, the set number of doses is determined using an artificial intelligence enabled PRS (Al-PRS) platform.

In still another embodiment, the administering step comprises administering at least one does of the vasopressor and at least one dose of the blood pressure reducing agent.

In a yet further embodiment, the vasopressor is selected from the group consisting of phenylephrine and norepinephrine.

In yet another embodiment, the blood pressure reducing agent is selected from the group consisting of nitroglycerin and fentanyl.

In a further embodiment again, the administering step further provides at least one dose of a positive inotrope, an inovasodilator, whole blood, a blood component, or a diuretic.

In another embodiment again, the individual is undergoing surgery and on anesthesia.

In a further additional embodiment, the individual being treated for a serious condition in the Intensive Care Unit or emergency room.

In another additional embodiment, the method further includes updating the PRS based on continually monitoring physiological responses to the administration of the at least one of the vasopressor or the blood pressure reducing agent.

In a still yet further embodiment, the controlling the blood pressure step is based on the physiological response described in the updated PRS.

In still yet another embodiment, an artificial intelligence enabled system for hemodynamic control includes a physiological monitor configured to measure at least one blood pressure component of an individual, at least one pump configured to administer at least one of a vasopressor or a blood pressure reducing agent to the individual, and a computing device in communication with the physiological monitor and the pump operating an artificial intelligence enabled phenotypic response surface (Al-PRS) platform, where the Al-PRS platform constructs a phenotypic response surface (PRS) describing the physiologic response of the individual in reaction to the at least one of the vasopressor or the blood pressure reducing agent and the computing device is configured to administer a dose of the vasopressor or the blood pressure reducing agent via the at least one pump upon a change in the at least one blood pressure component measured by the physiological monitor.

In a still further embodiment again, the at least one blood pressure component is selected from mean arterial pressure and left ventricular systolic pressure.

In still another embodiment again, the computing device is configured to maintain a target range of the at least one blood pressure component.

In a still further additional embodiment, the at least one blood pressure component is mean arterial pressure and the target range is 70±5 mmHg.

In still another additional embodiment, the computing device updates the PRS based on continually monitoring physiological responses to the administration of the at least one of the vasopressor or the blood pressure reducing agent.

In a yet further embodiment again, the at least one pump is at least two pumps, wherein a first pump is configured to administer a vasopressor and a second pump is configured to administer a blood pressure reducing agent.

In yet another embodiment again, the vasopressor is selected from the group consisting of phenylephrine and norepinephrine.

In a yet further additional embodiment, the blood pressure reducing agent is selected from the group consisting of nitroglycerin and fentanyl.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIGS. 1A-1B illustrate phenotypic response surfaces (PRSs) of drug responses in accordance with various embodiments.

FIG. 1C provides an Al-PRS equation in accordance with various embodiments.

FIGS. 2A-2B illustrate mean arterial blood pressure (MAP) as a function of time with boluses of medication indicated with a best-fit line of the MAP curve shown following medication administration in accordance with various embodiments.

FIG. 2C illustrates left ventricular systolic pressure (LVSP) under control of an artificial intelligence-enabled system in accordance with various embodiments.

FIG. 3 illustrates a system for artificial intelligence-enabled control of drug administration in accordance with various embodiments.

FIG. 4 illustrates a method for artificial intelligence-enabled control of drug administration in accordance with various embodiments.

### DETAILED DESCRIPTION OF THE INVENTION

Turning now to the drawings, systems and methods for artificial intelligence enabled control of hemodyamics and anesthesia in surgery patients are described. Many embodiments are directed to the use of artificial intelligence (Al) as a personalized medicine tool to tailor the anesthetic and hemodynamic management of patients based on their unique physiology and biochemistry response profiles. Various embodiments utilize Al-based phenotypic response surface (Al-PRS) platform to prospectively determine a patient's optimal drug and dose combination. (See e.g., U.S. Pat. Pub. No. 2014/0309974; the disclosure of which is incorporated by reference herein in its entirety.) The clinically validated Al-PRS platform of many embodiments optimizes medication dosing independent of underlying disease pathology through an inherent incorporation of a patient's unique pharmacokinetics and physiologic responses.

Embodiments of this disclosure are directed to identifying optimized combinations of input parameters for a complex system. The goal of optimization of some embodiments of this disclosure can be any one or any combination of reducing labor, reducing cost, reducing risk, increasing reliability, increasing efficacies, reducing side effects, reducing toxicities, and alleviating drug resistance, among others. In some embodiments, a specific example of treating diseases of a biological system with optimized drug combinations (or combinatorial drugs) and respective dosages is used to illustrate certain aspects of this disclosure. A biological system can include, for example, an individual cell, a collection of cells such as a cell culture or a cell line, an organ, a tissue, or a multi-cellular organism such as an animal, an individual human patient, or a group of human patients. A biological system can also include, for example, a multi-tissue system such as the nervous system, immune system, or cardio-vascular system.

More generally, embodiments of this disclosure can optimize wide varieties of other complex systems by applying pharmaceutical, chemical, nutritional, physical, or other types of stimulations. Applications of embodiments of this disclosure include, for example, optimization of drug combinations, vaccine or vaccine combinations, chemical synthesis, combinatorial chemistry, drug screening, treatment therapy, cosmetics, fragrances, and tissue engineering, as well as other scenarios where a group of optimized input parameters is of interest.

Stimulations can be applied to direct a complex system toward a desired state, such as applying drugs to treat a patient having a disease. The types and the values (e.g., amplitudes or dosages) of applying these stimulations are part of the input parameters that can affect the efficiency in bringing the system toward the desired state. However, N types of different drugs with M dosages for each drug will result in MN possible drug-dosage combinations. To identify an optimized or even near optimized combination by multiple tests on all possible combinations is prohibitive in practice. For example, it is not practical to perform all possible drug-dosage combinations in animal and clinical tests for finding an effective drug-dosage combination as the number of drugs and doses increase.

Embodiments of this disclosure provide a technique that allows a rapid search for optimized combinations of input parameters to guide multi-dimensional (or multi-variate) medical problems, as well as controlling other complex systems with multiple input parameters toward their desired states. An optimization technique can be used to identify at least a subset, or all, optimized combinations or sub-combinations of input parameters that produce desired states of a complex system. Taking the case of combinational drugs, for example, a combination of N drugs can be evaluated to rapidly identify optimized dosages of the N drugs, where N is greater than 1, such as 2 or more, 3 or more, 4 or more, 5 or more, 6 or more, 7 or more, 8 or more, 9 or more, or 10 or more.

For instance, in liver transplant patients receiving tacrolimus for post-transplant immunosuppression therapy, the utilization of Al-PRS, in accordance with some embodiments, resulted in significant reductions in the variation of tacrolimus trough levels compared to patients who received their tacrolimus doses based on a physician-guided regimen. (See e.g., A. Zarrinpar, Sci. Transl. Med. 8, 333ra49 (2016); the disclosure of which is incorporated by reference herein in its entirety.) The Al-PRS platform has also proved successful in the management of patients with prostate cancer through optimizing chemotherapy dosing, such as combinations of ZEN-3694 and enzalutamide, to minimize prostate specific antigen (PSA) levels. (See e.g., A. J. Pantuck, DOI: 10.100²/ₐdtp.201800104, Advanced Therapeutics, (2018), the disclosure of which is incorporated by reference herein in its entirety.)

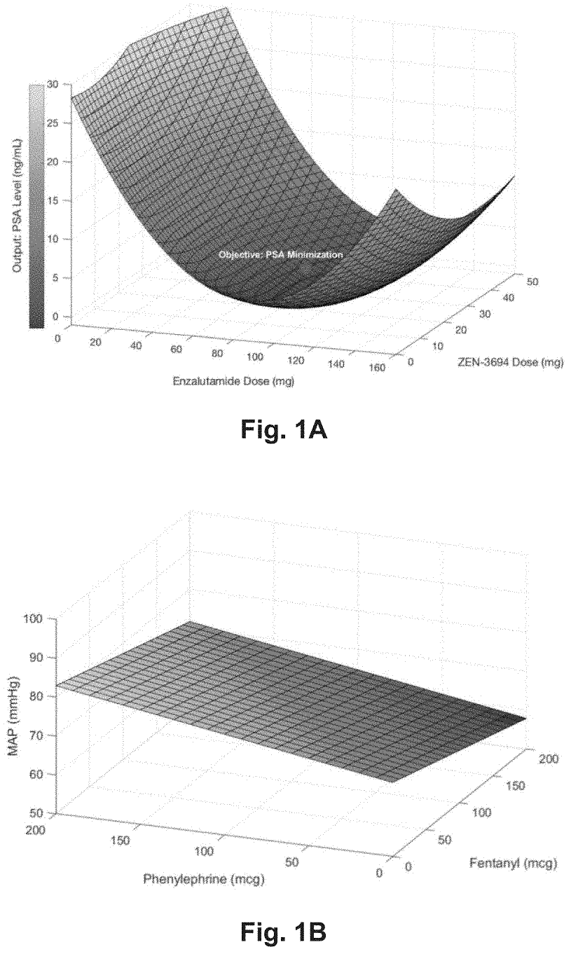

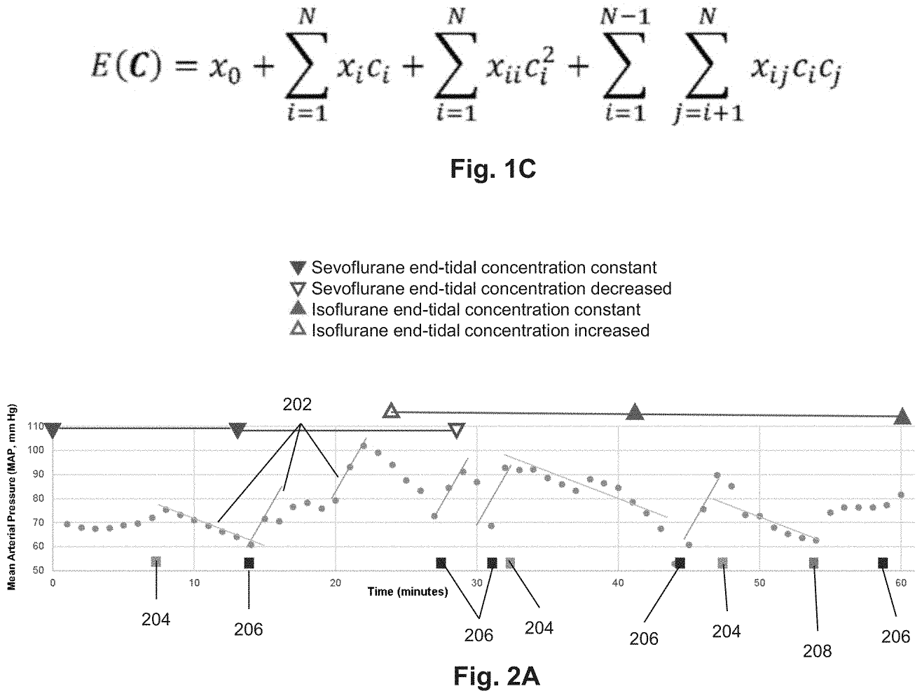

Al-PRS, as used in many embodiments, produces a three-dimensional smooth surface, referred to as the phenotypic response surface (PRS), and represents a patient's unique physiologic response to therapeutic agents, including but not limited to, blood pressure regulators (e.g., vasopressors and vasodilators), immunosuppressants, chemotherapeutics (including, but not limited to, ZEN-3694 and enzalutamide), anesthesia, and combinations thereof. For example, FIG. 1A illustrates an exemplary embodiment of a PRS profile of a patient with prostate cancer showing the appropriate doses of enzalutamide and ZEN-3694 to minimize the PSA level. Similarly, FIG. 1B illustrates an exemplary embodiment of a PRS profile depicting mean arterial blood pressure (MAP), a blood pressure component, response to phenylephrine and fentanyl administration. In many embodiments, the Al-PRS platform of many embodiments optimizes drug dosing to maximize treatment efficacy over physician-guided treatments. In many embodiments, the PRS is governed by the Al-PRS equation, as illustrated in FIG. 1C, where E(C) is MAP (Mean Arterial Pressure), Cᵢ is Dose of a vasopressor (e.g., phenylephrine) or vasodilator (e.g., nitroglycerin), or medication with effect of lowering blood pressure (e.g., fentanyl), and xo, xi, xii are experimental coefficients. Many embodiments incorporate a continuously learning Al platform to provide an individualized drug response and to guide choice of medication and dose to achieve control of one or more specific parameters.

Given the challenges of managing the hemodynamics of patients undergoing surgery or treatment in the ICU, embodiments utilize Al-PRS as a tool for medication selection and dose optimization. Many embodiments present Al-PRS as a personalized therapy and data-driven tool to tailor the anesthetic and hemodynamic management of patients in the operating room and ICU based on their unique physiology and biochemistry response profiles.

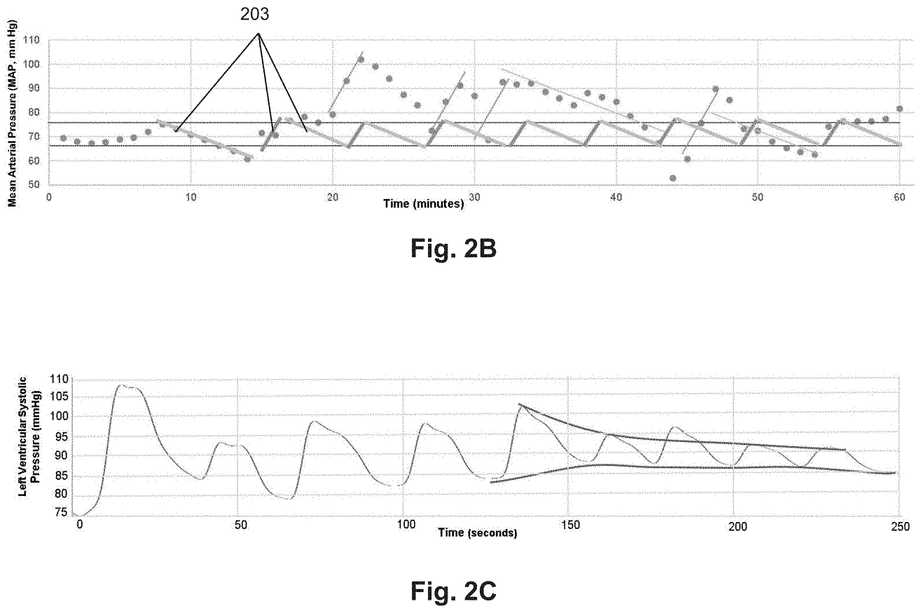

Turning to FIGS. 2A-2C, an exemplary embodiment of a hemodynamic control using Al-PRS is illustrated. In particular, FIG. 2A illustrates MAP plotted against time for a patient undergoing surgery. Trendlines 202 illustrate increasing and decreasing trends of the MAP over the time. Additionally, the timing of sevoflurane and isoflurane anesthetics are plotted in addition to the time of administration of boluses of fentanyl 204, phenylephrine 206, and norepinephrine infusion 208. Based on patient response to drug boluses, FIG. 2B illustrates an idealized level of control of MAP with trendlines 203 showing MAP control within a target range of approximately 65 mmHg and approximately 75 mmHg. However, the target range for blood pressure maintenance can be modified based on a patient. For example, some embodiments may maintain a mean target pressure of between 60-80±10 mmHg. Additionally, the target range may vary depending on the specific blood pressure component being measured (e.g., MAP versus LVSP). Additionally, FIG. 2C illustrates exemplary results of left ventricular systolic pressure (LVSP) control of an embodiment, where the blood pressure has decreased fluctuation under control of the embodiment.

### Systems for Hemodynamic Control

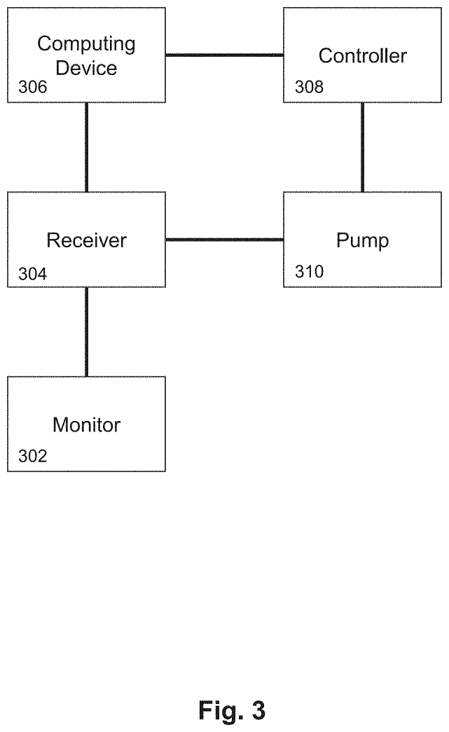

Turning to FIG. 3, various embodiments analyze hemodynamic data of patients undergoing cardiac surgery and use continuously learning Al platform to provide a patient's individual hemodynamic response to medications and to guide choice of medication as well as dose to achieve hemodynamic goals. Many embodiments possess a physiological monitor 302 to obtain physiological data about an individual under treatment. Certain embodiments monitor blood pressure and/or other clinical parameters. Certain embodiments utilize a monitor selected from GE Solar line, a LiDCo device, an Edwards Lifesciences EV-1000, and/or any other monitor known in the art.

Additional embodiments possess a receiver 304 in communication with and to obtain data from a monitor 302. Further embodiments comprise a computing device 306 comprising a processor and memory. A computing device 306 processes physiologic data coming from a receiver 304, such as to determine what medicine to administer (e.g., vasopressor or vasodilator), when to administer the medicine, and what dose to administer. In many embodiments, the computing device operates the Al-PRS platform to identify physiologic responses to a dose of medicine or drug and/or any interactions between multiple drugs. Such methods of determining dose using an Al-PRS platform in accordance with many embodiments is described elsewhere herein.

Further embodiments comprise one or more controllers 308 in communication with a computing device 306. Controllers 308 of many embodiments are configured to select a particular drug, timing, and/or dose as determined by a computing device 306. Further embodiments administer a medicine or drug via a pump 310, such as a peristaltic pump or any other pump sufficient for administering a particular drug based on timing of dose, rate of administration, and size of dose. In many embodiments, one or more pumps 310 are in communication with a receiver 304, such that the receiver further receives data regarding which medicine, dose amount, timing of dose, rate of administration of a dose, and/or any other information that a pump may have regarding medicine or drug administration. In such embodiments, a computing device 306 can further correlate dosing data with physiologic data received from a physiologic monitor 302.

It is known in the art that certain configurations may be combined into a single device, rather than individualized components, such that certain embodiments are contained as a single, integrated computing device comprising a monitor and pump, such that psychological data from a monitor is communicated directly to a processor, which further controls one or more integrated pumps to administer one or more drugs to a patient.

### Methods for Treating an Individual

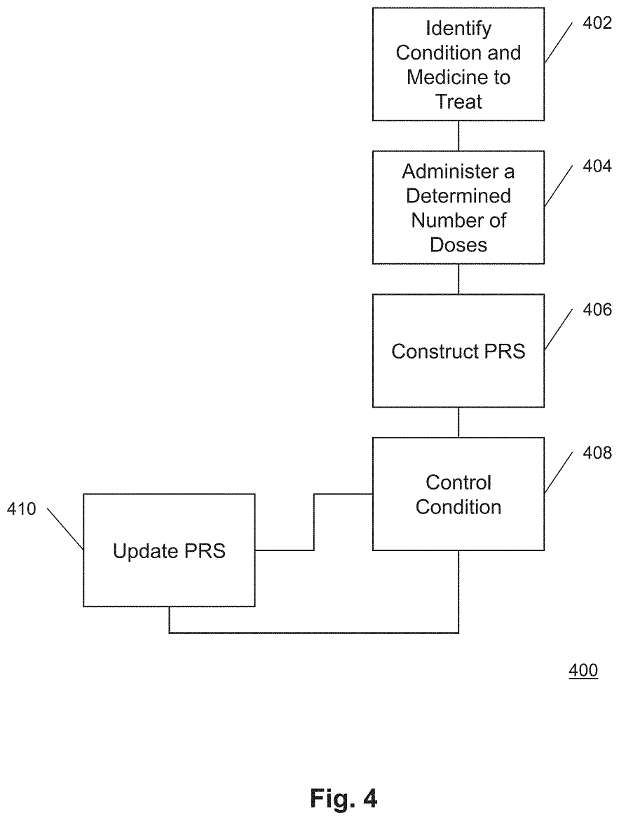

Turning to FIG. 4, some embodiments are directed to methods 400 to treat an individual based on an individual's physiological response to one or more drugs, including interactions between these drugs. At 402 of many embodiments, a treatable condition is identified along with one or more drugs for treatment. In some embodiments, the treatable condition is management of a disease, disorder, or physiological condition, such as a cancer, infections (e.g., viral and/or bacterial), blood pressure, diabetes, psychological/psychiatric disorders, and/or pain. Cancers include cancers of the bladder, prostate, breast, or any other form of cancer, while infections include viral infections such as HIV, Herpes simplex 1, Herpes simplex 2, coronaviruses, including SARS-CoV-2, and other viruses, while bacterial infections include pneumococcal bacteria, tuberculosis, and other chronic bacterial infections. Additionally, blood pressure management can include during surgical procedures, Intensive Care Unit admission, or whenever a patient becomes hemodynamically unstable where control of a target blood pressure component (e.g., MAP) is useful for better recovery from the surgical procedure. Additionally, certain embodiments identify pain disorders for treatment or management. Drugs for treatment of the condition include any relevant drug or combination of drugs for treatment, such as anti-inflammatories, antivirals, antibiotics, antipsychotics, vasopressors, vasodilators, blood pressure reducing agent, anesthesia, narcotics, opioids, insulin, steroids, any other drug relevant for treatment, and combinations thereof. In certain embodiments, the condition is controlling blood pressure of an individual during surgery, where the drugs consists of a vasopressor and a blood pressure reducing agent (e.g., vasodilator or drug with effect of reducing blood pressure). In some of these embodiments the vasopressor is selected from the group consisting of phenylephrine, norepinephrine and the blood pressure reducing agent is selected from the group consisting of nitroglycerin and fentanyl. Addition embodiments include administration of blood products, intravenous fluids, pain medication, positive inotrope (to increase myocardial contractility), inovasodilator (to increase myocardial contractility and increase vasodilation), a diuretic (to get rid of excess fluid) any other treatment for a condition being monitored, and combinations thereof.

At 404, many embodiments administer a determined number (or set) of doses to an individual. In some embodiments, the timing, dose size, and which drug to administer is determined via an Al-PRS platform, such as described herein and in U.S. Pat. Pub. No. 2014/0309974, cited above. An Al-PRS platform of many embodiments produces a PRS curve for an individual, based on physiologic response to the one or more drugs to be administered to an individual. Depending on the number of drugs or medicines to provide, the amount of data points to produce a PRS varies. As noted elsewhere herein, the Al-PRS platform of some embodiments is based on the equation illustrated in FIG. 1C.

At 406, many embodiments construct a PRS based on physiologic response to the one or more drugs. In many embodiments, the PRS is constructed using the Al-PRS platform upon administering the one or more drugs are administered to an individual and measuring the physiological response produced by the one or more drugs. In many embodiments, the PRS identifies the physiological response of the one or more drugs as well as any interactions between drugs being administered that affect the efficacy of one or more of the drugs.

At 408, further embodiments control of a condition identified in 402 with the drug or drugs also identified in 402, based on the PRS constructed in 406. Control in accordance with certain embodiments involves administering one or more of the drugs to produce its respective physiological response in the individual to maintain control of the condition or disease. In some embodiments, control is maintaining a target parameter within a specific range (e.g., MAP of 70±5 mmHg), while other embodiments control means maintaining a maximum or minimum parameter such as viral load or PSA.

Many embodiments update the PRS at 410 by continually monitoring physiological responses to the administration of the one or more drugs to the individual, which allow for continually improving control over the condition at 408.

It should be noted that certain embodiments may combine some features, repeat some features, or omit some features of method 400, as necessary for a particular purpose, such that controlling a condition, where multiple administrations of the one or more drugs may be necessary to maintain control of the condition.

### Exemplary Embodiments

Although the following embodiments provide details on certain embodiments of the inventions, it should be understood that these are only exemplary in nature, and are not intended to limit the scope of the invention.

### Example 1: Prospective Study on Rats

METHODS: In this embodiment, rats were anesthetized with the volatile anesthetic isoflurane and placed on mechanical ventilation via a tracheostomy. Venous access was obtained via the femoral vein and left ventricular systolic pressure (LVSP), a surrogate for systolic arterial pressure (SAP), was measured via a catheter that was placed through an incision in the left ventricle after sternotomy. LVSP was measured at baseline and following administration of medications to increase blood pressure (e.g., phenylephrine) or decrease blood pressure (e.g., nitroglycerin).

RESULTS: FIG. 1B illustrates a three-dimensional phenotypic response surface (PRS) depicting mean arterial blood pressure (MAP) response to phenylephrine and fentanyl administration.

CONCLUSION: This embodiment illustrates the ability of an embodiment to model physiologic response to a vasodilator and a vasopressor under anesthesia, such as a person undergoing surgery.

### DOCTRINE OF EQUIVALENTS

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above, and of the corresponding application(s), are hereby incorporated by reference.

What is claimed is:

1. A method for artificial intelligence enabled control of hemodyamics, comprising:
administering at least one of a vasopressor or a blood pressure reducing agent to an individual;
constructing a phenotypic response surface (PRS) describing the physiologic response of the individual in reaction to the at least one of the vasopressor or the blood pressure reducing agent; and
controlling the blood pressure of the individual by administering at least one dose of the vasopressor or the blood pressure reducing agent based on the physiological response described in the PRS.

2. The method of claim 1, further comprising updating the PRS based on additional physiological response to the at least one of the vasopressor or the blood pressure reducing agent.

3. The method of claim 1, wherein the administering step is provides a set number of doses of the at least one of the vasopressor or the blood pressure reducing agent.

4. The method of claim 3, wherein the set number of doses is determined using an artificial intelligence enabled PRS (Al-PRS) platform.

5. The method of claim 3, wherein the administering step comprises administering at least one does of the vasopressor and at least one dose of the blood pressure reducing agent.

6. The method of claim 1, wherein the vasopressor is selected from the group consisting of phenylephrine and norepinephrine.

7. The method of claim 1, wherein the blood pressure reducing agent is selected from the group consisting of nitroglycerin and fentanyl.

8. The method of claim 1, wherein the administering step further provides at least one dose of a positive inotrope, an inovasodilator, whole blood, a blood component, or a diuretic.

9. The method of claim 1, wherein the individual is undergoing surgery and on anesthesia.

10. The method of claim 1, wherein the individual being treated for a serious condition in the Intensive Care Unit or emergency room.

11. The method of claim 1, further comprising updating the PRS based on continually monitoring physiological responses to the administration of the at least one of the vasopressor or the blood pressure reducing agent.

12. The method of claim 11, wherein the controlling the blood pressure step is based on the physiological response described in the updated PRS.

13. An artificial intelligence enabled system for hemodynamic control, comprising:
a physiological monitor configured to measure at least one blood pressure component of an individual;
at least one pump configured to administer at least one of a vasopressor or a blood pressure reducing agent to the individual; and
a computing device in communication with the physiological monitor and the pump operating an artificial intelligence enabled phenotypic response surface (Al-PRS) platform;
wherein the Al-PRS platform constructs a phenotypic response surface (PRS) describing the physiologic response of the individual in reaction to the at least one of the vasopressor or the blood pressure reducing agent; and
wherein the computing device is configured to administer a dose of the vasopressor or the blood pressure reducing agent via the at least one pump upon a change in the at least one blood pressure component measured by the physiological monitor.

14. The system of claim 13, wherein the at least one blood pressure component is selected from mean arterial pressure and left ventricular systolic pressure.

15. The system of claim 13, wherein the computing device is configured to maintain a target range of the at least one blood pressure component.

16. The system of claim 15, wherein the at least one blood pressure component is mean arterial pressure and the target range is 70±5 mmHg.

17. The system of claim 11, wherein the computing device updates the PRS based on continually monitoring physiological responses to the administration of the at least one of the vasopressor or the blood pressure reducing agent.

18. The system of claim 11, wherein the at least one pump is at least two pumps, wherein a first pump is configured to administer a vasopressor and a second pump is configured to administer a blood pressure reducing agent.

19. The system of claim 11, wherein the vasopressor is selected from the group consisting of phenylephrine and norepinephrine.

20. The system of claim 11, wherein the blood pressure reducing agent is selected from the group consisting of nitroglycerin and fentanyl.
